(19) United States
(12) Patent Application Publication
(10) Pub. No.: US 2023/0218893 A1
(43) Pub. Date: Jul. 13, 2023

(54) Code-Controlled Multi-Site Wirelessly-Powered Batteryless Stimulator

(71) Applicant: The Regents of the University of California, Oakland, CA (US)

(72) Inventors: Aydin Babakhani, Los Angeles, CA (US); Iman Habibagahi, Los Angeles, CA (US)

(73) Assignee: The Regents of the University of California, Oakland, CA (US)

(21) Appl. No.: 18/153,869

(22) Filed: Jan. 12, 2023

Related U.S. Application Data
(60) Provisional application No. 63/266,738, filed on Jan. 13, 2022.

(51) Int. Cl. A61N 1/05; A61N 1/36; H02J 50/00; H02J 50/12; H02J 50/80

(52) U.S. Cl. CPC A61N 1/0551; A61N 1/36153; H02J 50/001; H02J 50/12; H02J 50/80; H02J 2310/23

(57) ABSTRACT

Wirelessly powered and controlled implantable stimulator system in accordance with embodiments of the invention are described. One embodiment includes: a transmitter (TX) coil wirelessly powering and controlling several implantable stimulators though electromagnetic waves that include modulated waveforms that include n-bit passcodes to individually control stimulation of each of the plurality of implantable stimulators; where an implantable stimulator of the several implantable stimulators includes: a receiver (RX) for receiving a modulated waveform from the TX coil, where the implantable stimulator is controlled based on the modulated waveform.

### CROSS REFERENCE TO RELATED APPLICATIONS

This application claims benefit of and priority under 35 U.S.C. 119(e) to U.S. Provisional Patent Application No. 63/266,738 entitled “Code-Controlled Multi-Site Wirelessly-Powered Batteryless Stimulator” by Babakhani et al., filed Jan. 13, 2022, the disclosure of which is hereby incorporated by reference in its entirety for all purposes.

### STATEMENT OF FEDERALLY SPONSORED RESEARCH

This invention was made with government support under Grant Number HL144683, awarded by the National Institutes of Health. The government has certain rights in the invention.

### FIELD OF THE INVENTION

The present invention generally relates to code-controlled multi-site wirelessly-powered batteryless stimulators.

### BACKGROUND

Multisite stimulation has proven helpful for different medical applications. For example, in mice, it has been shown that different stimulation patterns in the spinal cord could lead to unique muscle activation. It has also been shown that multisite spinal cord stimulation can enable faster recovery of motor functions. The efficacy of multisite stimulation has been proven for enhanced cardiac resynchronization therapy as well. Both unilateral and bilateral VNS have proven to be useful for cardiac therapy, epilepsy treatment, and weight control. From the point of care perspective, miniaturized and battery-free implants are preferred since natural movements are maintained, and chances of infections or reoperation can be minimized.

There have been multiple approaches to implementing multisite stimulation. As shown in FIG. 1A, for spinal cord stimulation, prior techniques can include using electrode arrays to stimulate different nerves. However, this approach may not be easily scalable as the implant size and length of electrodes increase significantly with the number of stimulation sites. As illustrated in FIG. 1B, prior techniques have included biventricular heart pacing with two implants operating with two carrier frequencies. A drawback of this approach is that interference and unwanted couplings between Tx coils limit the operation distance. Furthermore, the number of Tx coils scales linearly with the number of implants, which shows the undesired scaling of the system. FIG. 1C illustrates a prior technique to perform multisite stimulation, where physical unclonable function (PUF) IDs were implemented to address each implant individually. This approach may benefit from good scalability; however, PUFs have inherent instability and sensitivity to process voltage, and temperature variations (PVT). Complex circuitry based on temporal majority voting (TMV) may need to be adopted to improve stability. Furthermore, additional mask layers may need to be used in the fabrication process to implement Native NMOS transistors for PUFs which leads to higher fabrication costs.

### SUMMARY OF THE INVENTION

Systems and methods for code-controlled multi-site wirelessly-powered batteryless stimulators in accordance with embodiments of the invention are described. An embodiment includes a wirelessly powered and controlled implantable stimulator system, including: a transmitter (TX) coil wirelessly powering and controlling several implantable stimulators though electromagnetic waves, where the electromagnetic waves include modulated waveforms that include n-bit passcodes to individually control stimulation of each of the several implantable stimulators; where an implantable stimulator of the several implantable stimulators includes: a receiver (RX) for receiving a modulated waveform from the TX coil, where the implantable stimulator is controlled based on the modulated waveform; a coil that harvests AC power; a voltage regulator; a rectifier that rectifies the harvested power and passes the rectified voltage (Vrect) to the voltage regulator to provide a constant voltage; data recovery and control circuitry coupled to the voltage regulator and configured to output a stimulation control signal based on passcodes detected from the modulated waveform; an output stage and driver circuitry coupled to receive the constant voltage from the voltage regulator and the stimulation control signal from the data recovery and control circuitry, and configured to output a stimulation signal; and one or more electrodes coupled to receive the stimulation signal from the output stage and driver circuitry.

In a further embodiment, an n-bit passcode is hard-wired and set on a printed circuit board (PCB) for each of the several implantable stimulators, where the modulated waveform includes an n-bit passcode to communicate with a particular implantable stimulator of the several implantable stimulators, where the modulated waveform includes several stimulation control settings including a start/stop signal and a voltage level of a stimulation pulse to be delivered by an implantable stimulator.

In a further embodiment, the modulated waveform includes several bits, where a first set of bits of the several bits address a particular implantable stimulator from the several stimulators, at least one bit of the several bits determines a voltage level of a stimulation, and at least one bit of the several bits determines a start/stop signal of a stimulation.

In a further embodiment again, the TX coil wirelessly powers and controls the several implantable stimulators through a near field resonant inductive link.

In a further embodiment again, the modulated waveform is a pulse-width modulated amplitude-shift keying (PWM-ASK) waveform.

In a further embodiment again, the Vrect drops below a threshold voltage and a control block stops stimulation.

In a further embodiment again, at least one implantable stimulator in the several implantable stimulators is battery-less such that the implantable stimulator is wirelessly powered and memory-less such that control of a stimulation of the at least one implantable stimulator is controlled based on the modulated waveform received from the TX coil.

In a further embodiment again, each implantable stimulator in the several implantable stimulators is individually addressable using an n-bit passcode.

In a further embodiment again, the implantable stimulator further includes a system on chip (SOC), wherein the SOC includes: a power management unit (PMU); a clock and data recovery (CDR) unit that recovers clock and data from the modulated waveform; an envelope detection unit; an error detection unit; and an output driving stage.

In a further embodiment again, the voltage regulator is a low dropout voltage regulator (LDO) that includes an amplifier in a negative feedback loop.

In a further embodiment again, the Vrect exceeds a threshold voltage and a limiter is activated and stops the Vrect from accumulating further.

In a further embodiment again, the rectifier is a fully differential cross-coupled 4-stage rectifier.

In a further embodiment again, the RX is used to extract both clock and data, where an incoming signal is self-mixed using the rectifier and low-pass filtered and passed through a Schmitt trigger to extract its envelope that includes baseband data.

In a further embodiment again, clock and data are recovered by passing the envelope though an integrator and a comparator.

In a further embodiment again, the implantable stimulator further includes a finite state machine (FSM) that controls a stimulation pulse width and amplitude of stimulation.

In a further embodiment again, the implantable stimulator is controlled by a 4-bit passcode.

Another embodiment includes a medical system including: a transmitter (TX) coil configured to output transmitter waves having a pulse-width and an amplitude; and several implantable stimulators, each implantable stimulator including: a coil configured to establish a near-field resonant inductive link with the transmitter coil, and to harvest power from the transmitter waves; a voltage regulator that outputs a constant voltage; data recovery and control circuitry coupled to the voltage regulator and configured to output a stimulation control signal based on passcodes detected from the transmitter waves; an output stage and driver circuitry coupled to receive the constant voltage from the voltage regulator and the stimulation control signal from the data recovery and control circuitry, and configured to output a stimulation signal; and one or more electrodes coupled to receive the stimulation signal from the output stage and driver circuitry.

In a further embodiment again, each implantable stimulator further includes: a rectifier that rectifies the harvested power and passes the rectified voltage (Vrect) to the voltage regulator to provide the constant voltage; where output stage and driver circuitry is configured to stop stimulation in response to the Vrect dropping below a threshold voltage.

In a further embodiment again, at least one of the several implantable stimulators is configured for implant in or on a heart, and the stimulation signal is configured to evoke a depolarization of the heart.

In a further embodiment again, a first of the several implantable stimulators is configured for implant in or on a left ventricle of a heart, a second of the several implantable stimulators is configured for implant in or on a right ventricle of a heart, and the output stage and driver circuitry is configured to output respective stimulation signals to the first implantable stimulator and the second implantable stimulator to deliver biventricular pacing.

In a further embodiment again, at least one of the several implantable stimulators is configured for implant in or on a brain, and the stimulation signal is configured to elicit a neural effect.

In a further embodiment again, at least one of the several implantable stimulators is configured for implant in or on a spine, and the stimulation signal is configured to elicit a neural effect.

In a further embodiment again, a first of the several implantable stimulators is configured for implant on a first side of a neck to stimulate a right vagus nerve, a second of the several implantable stimulators is configured for implant on a second side of the neck to stimulate a left vagus nerve, and the output stage and driver circuitry is configured to output respective stimulation signals to the first implantable stimulator and the second implantable stimulator.

In a further embodiment again, the nerve is a vagal nerve.

In a further embodiment again, the transmitter (TX) coil is included in a component configured to be implanted in operation vicinity (within about 80 mm) of each of the several implantable stimulators.

In a further embodiment again, the transmitter (TX) coil is included in an external component configured to be placed in operation vicinity (within about 80 mm) of each of the several implantable stimulators.

In a further embodiment again, at least one of the several implantable stimulators is configured for implant in, on, or adjacent to a peripheral nerve.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

The patent or application file contains at least one drawing executed in color. Copies of this patent or patent application publication with color drawing(s) will be provided by the Office upon request and payment of the necessary fee.

FIG. 1A illustrates prior techniques for electrode array multisite stimulation.

FIG. 1B illustrates prior techniques for double carrier frequency for cardiac pacing.

FIG. 1C illustrates prior techniques for PUF enabled IDs.

FIG. 1D illustrates miniaturized implants based on printed circuit board (PCB)-defined passcodes in accordance with an embodiment of the invention.

FIG. 2 illustrates a battery-less implant for bilateral VNS with a miniaturized circular coil in accordance with an embodiment of the invention.

FIG. 3 illustrates inductive wireless power transfer link model: (a) schematics and (b) physical size of coils, in accordance with an embodiment of the invention.

FIG. 4 illustrates dielectric characteristics of different biological tissues: item (a) illustrates electrical permittivity and item (b) illustrates electrical conductivity in accordance with an embodiment of the invention.

FIG. 5 illustrates effect of biological tissue on Rx coil resonance: (a) modeled implant inside the neck in HFSS; and (b) drift in resonance frequency shift after adding biological tissue in accordance with an embodiment of the invention.

FIG. 6 illustrates simulated SAR over the human body in ANSYS HFSS with 1-W Tx coil in accordance with an embodiment of the invention.

FIG. 7 illustrates a circuit architecture of an implant in accordance with an embodiment of the invention.

FIG. 8 illustrates (a) 4-stage rectifier with voltage limiter and (b) one stage of CC rectifier in accordance with an embodiment of the invention.

FIG. 9 illustrates variation of a rectifier with respect to scaling and number of stages: (a) BW (b) Q_{ηrect} in accordance with an embodiment of the invention.

FIG. 10 illustrates variation of a rectifier with respect to input power and loading: (a) Rᵢₙ (b) Cᵢₙ (c) f_{Res} in accordance with an embodiment of the invention.

FIG. 11 illustrates a procedure for co-designing rectifier and Rx coil for reliable operation in accordance with an embodiment of the invention.

FIG. 12 illustrates a circuit schematic of an LDO in accordance with an embodiment of the invention.

FIG. 13 illustrates schematics of POR for digital blocks in accordance with an embodiment of the invention.

FIG. 14 illustrates a block diagram for clock and data recovery: (a) clock recovery chain (b) data recovery chain in accordance with an embodiment of the invention.

FIG. 15 illustrates a schematic of a reference generator, comparator, and Schmitt trigger in accordance with an embodiment of the invention.

FIG. 16 illustrates Monte Carlo simulation of a recovered clock frequency in accordance with an embodiment of the invention.

FIG. 17 illustrates a timing diagram of a stimulator chip: (a) conceptual presentation and (b) FSM in accordance with an embodiment of the invention.

FIG. 18 illustrates an output and control stage of a stimulator in accordance with an embodiment of the invention.

FIG. 19 illustrates a micrograph of an Implant's system-on-chip (SoC) in accordance with an embodiment of the invention.

### DETAILED DESCRIPTION OF THE DRAWINGS

Turning now to the drawings, systems and methods for code-controlled multi-site wirelessly-powered batteryless stimulators in accordance with embodiments of the invention are described. Implantable bioelectronics have been clinically used for a wide range of indications. Multisite stimulations have shown to enhance clinical outcomes in different applications such as, multi-chamber cardiac pacing, e.g., biventricular cardiac pacing, and neural stimulation, e.g., spinal cord stimulation, brain stimulation, and bilateral vagus nerve stimulation (VNS) among other applications. Wirelessly powered and controlled implantable devices in accordance with many embodiments can improve miniaturization and safety. In applications such as cardiac pacing or VNS in big animals, the volume of an implant can become unavoidably large when the stimulation sites are far apart from each other and where there is a need for long leads to connect electrodes to the stimulation site. As described, multiple carrier frequencies for implants has been proposed as an alternative solution. However, it has limitations due to the interference between the channels and the scalability of the system due to the higher number of Tx coils. Physical unclonable functions (PUF) have been introduced. However, the PUF may reduce robustness in the presence of thermal noise and supply variations. Furthermore, a PUF can have high complexity and fabrication costs due to native layers. Existing wireless power transfer techniques such as magnetoelectric (ME) or ultrasonic are not currently available for large animals because of the limited distance between Tx and implant (e.g., 40 mm). Moreover, Ultrasonic and ME based devices suffer from narrow channel bandwidths for data communication.

To address the challenges mentioned above, implantable stimulation systems in accordance with many embodiments of the invention provide a hardware platform with a single Tx coil wirelessly powering and controlling multiple implants (e.g., ≤16) through a near-field resonant inductive link. Stimulation systems in accordance with many embodiments can have one or more of the following specifications: (1) robust multi-channel (e.g., 2 channel, 1.8 V or 3.3 V) stimulation for each implant; (2) wirelessly powering and controlling up to a needed long lead distance (e.g., 80 mm in distance); (3) controlling up to N implants (e.g., 16 implants) using an n-bit passcode (e.g., 4-bit passcode); (4) controlling several and/or all implants using a single Tx coil and up to a certain needed distance and misalignment (e.g., 80 mm distance and 70° misalignment between Tx and implants) can be tolerated; (5) miniaturized implants can have a minimal volume and weight as beneficial for use as implants (e.g., volume of 20.1 mm3 and weigh approx., 80 mg).

Stimulation systems in accordance with many embodiments can include an implant that can include a multi-turn coil, storage capacitor, discharge resistor, one or more electrodes (planar, ring, cuff, etc.) and a System on Chip (SoC). In many embodiments, the SoC can include a power management unit (PMU), clock and data recovery (CDR), and output driving stage. The AC power can be harvested from the coil and can be rectified using a fully differential cross-coupled 4-stage rectifier. The rectifier voltage (Vrect) can be then passed to an LDO to provide a constant voltage (e.g., 1.8V (VDD) voltage). The LDO can be a compensated two-stage amplifier in a negative feedback loop.

In many embodiments, if the rectifier voltage drops below a certain voltage (e.g., 2.6V) an error detection circuitry in a control block can stop a stimulation. If the rectifier voltage exceeds a certain voltage (e.g., 3.8V) the limiter can activate and stop the voltage from accumulating further. A finite state machine (FSM) can control the stimulation pulse width and amplitude of stimulation.

Many embodiments of the system can include an implant with a System on Chip (SOC) that can generate several different voltage levels of stimulation (e.g., two voltage level stimulation) of several implants (e.g., 16 implants) using a single Tx coil. Each implant can operate at up to a particular distance (e.g., of about 80 mm) in the air through an inductive resonant link. In many embodiments, an SoC can consume minimal static power (e.g., only approximately 27 μW static power) and enable several channels of stimulation amplitude (e.g., two channels with stimulation amplitudes of 1.8 V and 3.3 V) and a particular timing resolution (e.g., timing resolution of 100 μs). An SoC can be implemented in the standard 180 nm CMOS technology and can have a minimal area (e.g., minimal area of approximately 0.75 mm×1.6 mm). In many embodiments, an SoC can include a RF rectifier, low drop-out regulator (LDO), error detection block, clock data recovery, finite state machine (FSM), and output stage. Each implant can have a PCB-defined passcode, which can enable the individual addressability of the implants for synchronized therapies. For example, an implantable device can weigh only about 80 mg and sizes about 20.1 mm³. Tolerance of up to 70° to angular misalignment was measured at a distance of 50 mm in a phosphate buffered solution (PBS). Many embodiments of the implantable stimulation system can be used for bilateral stimulation including bilateral vagus nerve stimulation (VNS) among others.

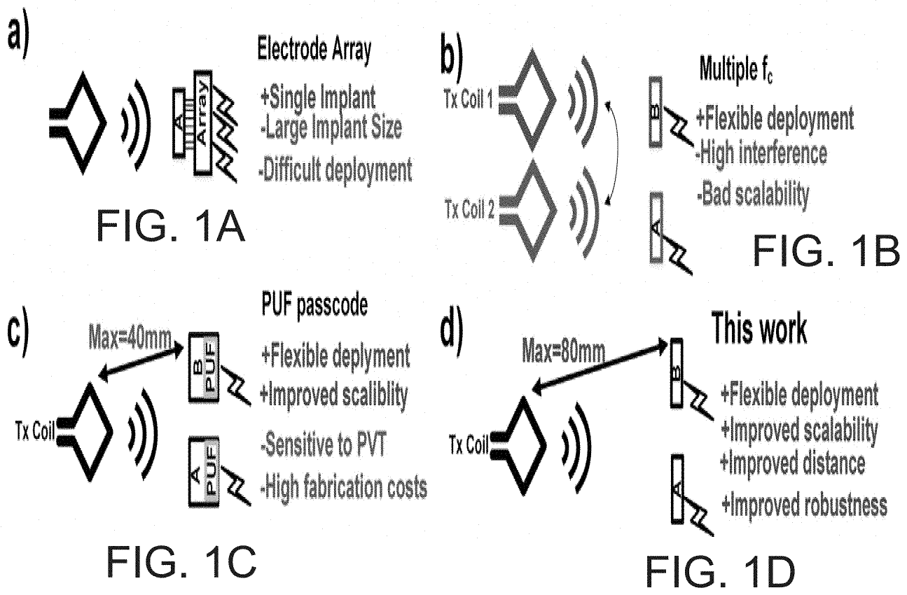

In many embodiments, a passcode-enabled implant can be controlled by at least one Tx coil. A passcode-enabled miniaturized implant controlled by a single Tx coil in accordance with an embodiment of the invention is illustrated in FIG. 1D.

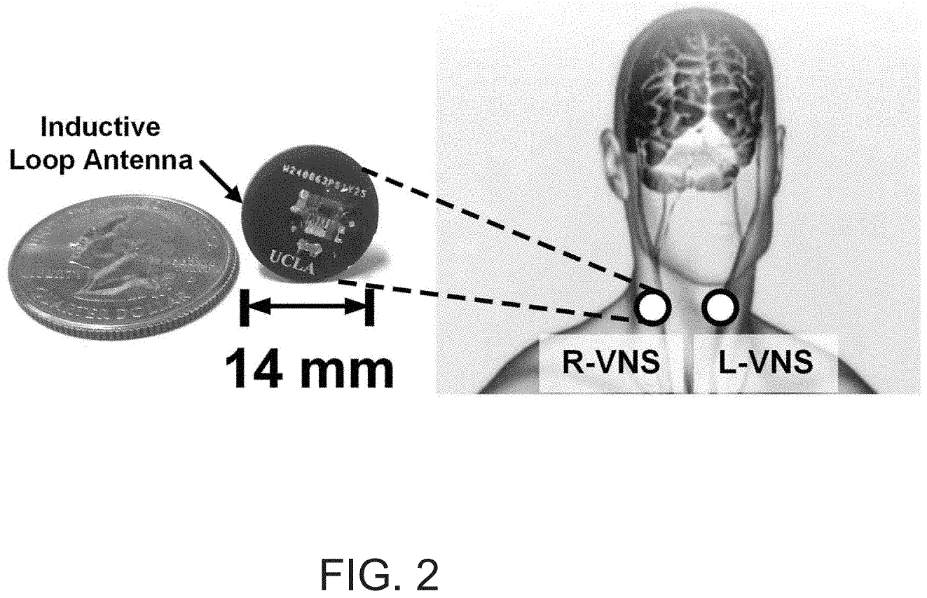

In many embodiments, an n-bit (e.g., 4-bit) passcodes can be defined on a chip's pads. Depending on the printed circuit board (PCB) connections, they can be connected to VDD or VSS. This approach can provide a robust passcode for individual addressability. In many embodiments, the Tx coil can power all the implants simultaneously, and by using Pulse-width modulated amplitude-shift keying (PWM-ASK), implants can be synchronized. Since each implant can be deployed at a different site, there may no longer be a need for long leads and/or electrode arrays. By optimizing link efficiency, a particular longer lead distance (e.g., of 80 mm) can be achieved by using a particular peak power (e.g., 2 W of peak power) for the Tx coil. Many embodiments of the implant can have: (1) reliable two-channel stimulation (e.g., 1.8V or 3.3V); (2) wirelessly powering and controlling up to a particular longer lead distance in air through a frequency link (e.g., 80 mm in air through inductive 40.68 MHz link); (3) an n-bit passcode which can enable N implants to operate at the same time (e.g., 4-bit passcode which enables up to 16 implants to operate at the same time; (4) up to about 70° angular misalignment can be tolerated; (5) implants fabricated using standard FR4 substrate technology can weigh approximately only 80 mg and have a volume of about 20.1 mm³; (6) error detection block can verify if an implant has harvested enough power before stimulation. A conceptual presentation of wirelessly powered and controlled implants for bilateral VNS with two implants in accordance with an embodiment of the invention is illustrated in FIG. 2.

Described herein are quantitative and qualitative details of a WPT link co-design and circuit implementation, coil designs, wireless link parameters, chip design, including rectifier, LDO, power-on reset (POR), CDR, and output stage, among other systems components that can be utilized as appropriate to the requirements of specific applications in accordance with embodiments of the invention.

### Wireless Link Designs

Stimulation systems in accordance with many embodiments can include magnetic resonance power transfer. Capacitive WPT can be sensitive to wireless link parameters. Ultrasound WPT can rely on physical vibrations for power transfer. A limitation of ultrasound can be that it may require direct physical contact by applied gel and can suffer from attenuation in muscle and bone mediums. A drawback of Magnetic resonance WPT can include large physical dimensions of a coil and tuning of a carrier frequency (f_{c}). Furthermore, wireless link variations and rectifier non-linearity can be pivotal factors for frequency tuning.

Coil Design

Implantable stimulator systems in accordance with many embodiments can include a matched series resonant Tx coil and parallel resonant Rx coil which can provide significantly lower power consumption on an implant side. This structure can have superior efficiency in low-power applications. In many embodiments, an approach for a coil design can involve an iterative process. A link efficiency can be calculated from the equation below:

$${η}_{link} \approx \frac{{K}^{2} {Q}_{T x} {Q}_{L}}{1 + {K}^{2} {Q}_{T x} {Q}_{L}} \times \frac{{Q}_{L}}{{Q}_{L} + {Q}_{R x}} \qquad ( 1 )$$

In equation (1), K is the distance-dependent coupling factor, Q_{Tx} is the Tx coil quality factor (Q_{Tx}=ωL_{Tx}/R_{Tx}), Q_{Rx} is the Rx coil quality factor (Q_{Rx}=ωL_{Rx}/R_{Rx}), and Q_{L} is the loaded Rx coil quality factor which can be calculated from the following equation:

$${Q}_{L} = \frac{1}{\frac{{R}_{R x}}{ω {L}_{R x}} + \frac{ω {L}_{R x}}{{R}_{L}}} \qquad ( 2 )$$

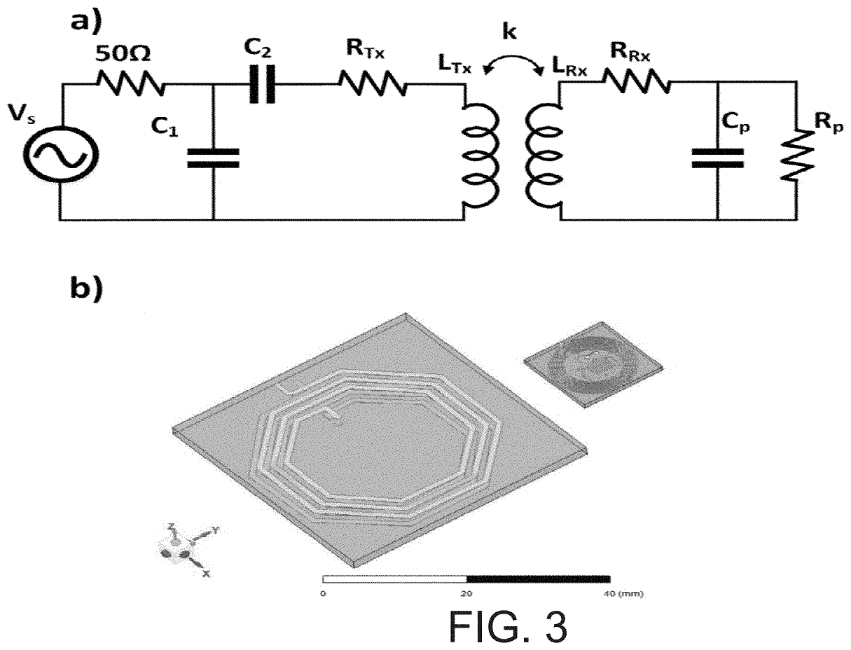

In many embodiments, a circular coil shape for Rx can be used due to its higher quality facto. A schematic of inductively coupled coils in accordance with an embodiment of the invention is illustrated in FIG. 3(a). The quality (Q) and the coupling factor (K) can be calculated from the number of turns (N), spacing between turns (S), trace width (W), and distance between coil (d). The following steps can be taken to design the Rx and Tx coil at a distance of 50 mm. For an initial design of the Tx coil, certain guidelines can be taken into account, such as equal width and spacing (W≈S), and the dimension of the optimal Tx coil (D) is less than the distance (d) from the following equation:

D≤2.288×d (3)

Step1: Apply constraints on the Rx and Tx coil size.

Step2: Initialize N, W, S for Tx coil.

Step3: Optimize Rx coil parameters for best ηₗᵢₙₖ.

Step4: Optimize Tx coil Parameters for best ηₗᵢₙₖ.

Step5: Repeat until there is no improvement.

For example, a simulated link efficiency in HFSS before matching can be 2.17% at a distance of 50 mm with an unmatched coil and R_{L}=100 kΩ. Physical dimensions of simulated coils in accordance with an embodiment of the invention are shown in HFSS. Details of coil stand-alone parameters in accordance with an embodiment of the invention are presented in table 1. A Tx coil can be connected to the N5230C network analyzer directly for characterization. The Rx coil, due to its small size and high parasitics of the probes, is not measured directly, and HFSS simulation results are shown in the table 1.

FIG. 3 illustrates an inductive wireless power transfer link model. (a) Schematics (b) Physical size of the designed coils.

TABLE 1
Tx and Rx coil parameters

| | | |
|---|---|---|
| Coil | Tx | Rx |
| parameters | (Measured) | (HFSS) |

| | | |
|---|---|---|
| Dimensions | 35 mm × | 14 mm |
| | 35 mm | diameter |
| No. Turns and | 6 and 2 | 12 and 2 |
| No. Layers | | |
| Q@f_{c} | 40.34 | 66.2 |

| | | | | |
|---|---|---|---|---|
| L@f_{c} | 1.96 | μH | 1.91 | μH |
| SRF | 58.82 | MHz | 90.44 | MHz |
| W and S | 1.1 | mm | 0.18 | mm |

### Wireless Link Characterization

Biological Medium Effects

Wireless link parameters such as power and medium variations can cause a change in an implant's resonant frequency. The intervening biological medium can be approximated as the low-loss complex dielectric material. The real and imaginary part of the dielectric constant corresponds to electrical permittivity and conductivity, respectively. The equation below expresses the frequency-dependent dielectric constant:

$$ϵ ( f ) = {ϵ}_{0} {ϵ}_{1} ( f ) + i {ϵ}_{2} ( f ) = {ϵ}^{′} ( f ) + i \frac{σ ( f )}{2 π f} \qquad ( 4 )$$

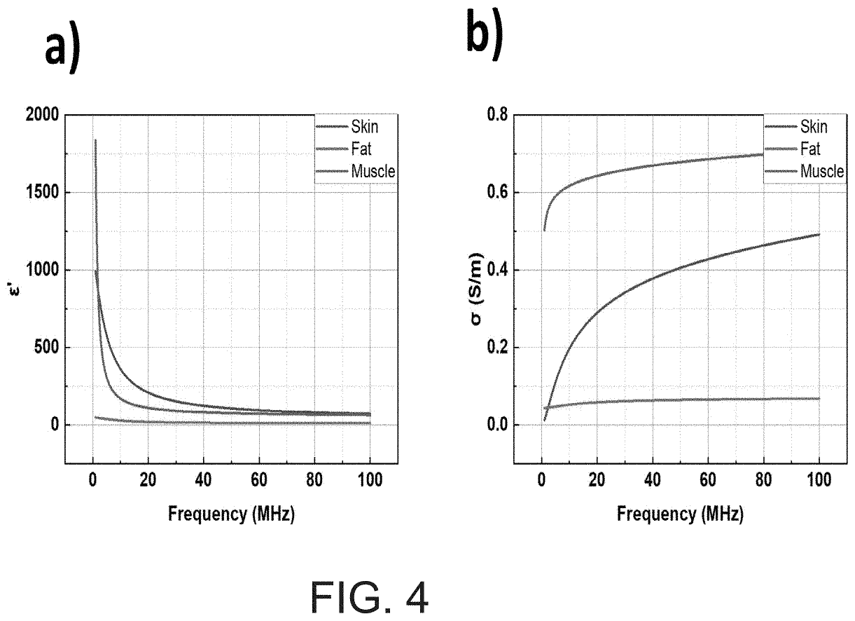

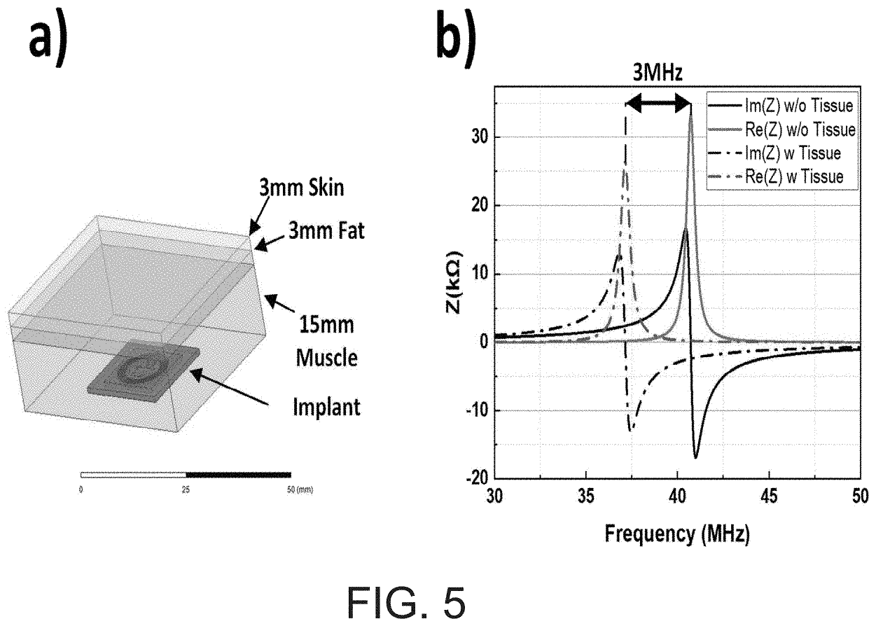

In frequencies of a few MHz and above, the dielectric conductivity increase and permittivity decrease can no longer be ignored. A database of dielectric properties of different biological tissues is available in the literature. The dielectric properties can be imported into HFSS to investigate the shift in resonance frequency after adding the material. Electrical permittivity and conductivity of skin, fat, and muscle are plotted in FIG. 4(a) and FIG. 4(b), respectively. Depending on the application and animal size, the biological medium can vary. In many embodiments, an implant can be placed inside an organism (e.g. pig's) neck for VNS. For example for a large animal, tissue can be modeled with 3 mm of skin, 3 mm of fat, and 15 mm of muscle on top of the implant, while the bottom of the implant is modeled as an infinite muscle due to the large size of the animal. Due to high electrical permittivity and conductivity, the effective capacitance of the coil increases, and the quality factor reduces after adding the biological tissue. The simulated implant inside the muscle and 3 MHz shift in resonance point is presented in FIG. 5 item (a) and FIG. 5 item (b), respectively. In particular, FIG. 5 illustrates effect of biological tissue on Rx coil resonance, with FIG. 5 item (a) illustrates a modeled implant inside the neck in HFSS and FIG. 5 item (b) illustrates drift in resonance frequency shift after adding biological tissue in accordance with an embodiment.

It can be possible to bring the resonance frequency back to 40.68 MHz by reducing Cₚ. Power transfer can be done at 40.68 MHz to operate within the industrial, scientific, and medical (ISM) band. In many embodiments, since an implant may need to work inside air and muscle properly, a system's bandwidth can be high enough to cover the frequency shift.

### Specific Absorption Rate

The specific absorption rate (SAR) can be an important figure of merit in determining how safe the wireless power transfer is to biological tissue. SAR can be calculated by the electric field inside the homogeneous tissue and can be calculated from the following equation:

$$S A R = \frac{σ \vert {E}_{r m s}^{2} \vert}{ρ} \qquad ( 5 )$$

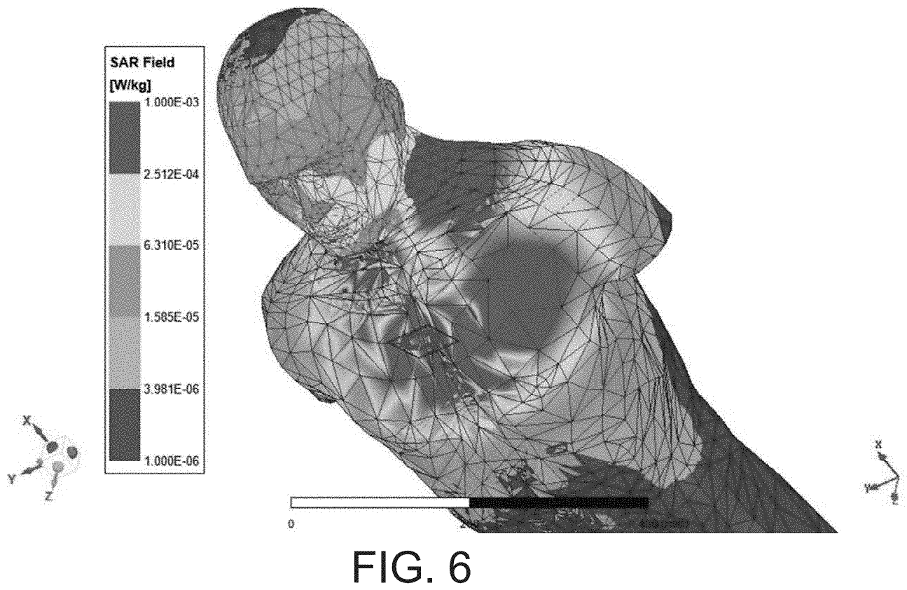

σ is the electrical conductivity, and ρ is the density of the tissue. As described, conductivity increases with frequency, which results in a higher absorption rate. Federal Communication Commission (FCC) recognizes the limit of 1.6 W/kg for the human head. The simulated specific absorption rate is 2 mW/kg in HFSS, which is three orders of magnitude less than the 10 W/kg safety limit according to IEEE Std C95.1-2005 and FCC regulation. The SAR can be simulated over the human model with a port power of 1 W at 40.68 MHz when the Tx coil is placed 3 cm away. FIG. 6 illustrates simulated SAR in HFSS averaged on the human body in accordance with an embodiment of the invention. Based on the calculated SAR and link efficiency calculated link efficiency it can be useful to limit the average power consumption of the chip to hundreds of micro-watts to ensure safety.

### Chip Designs

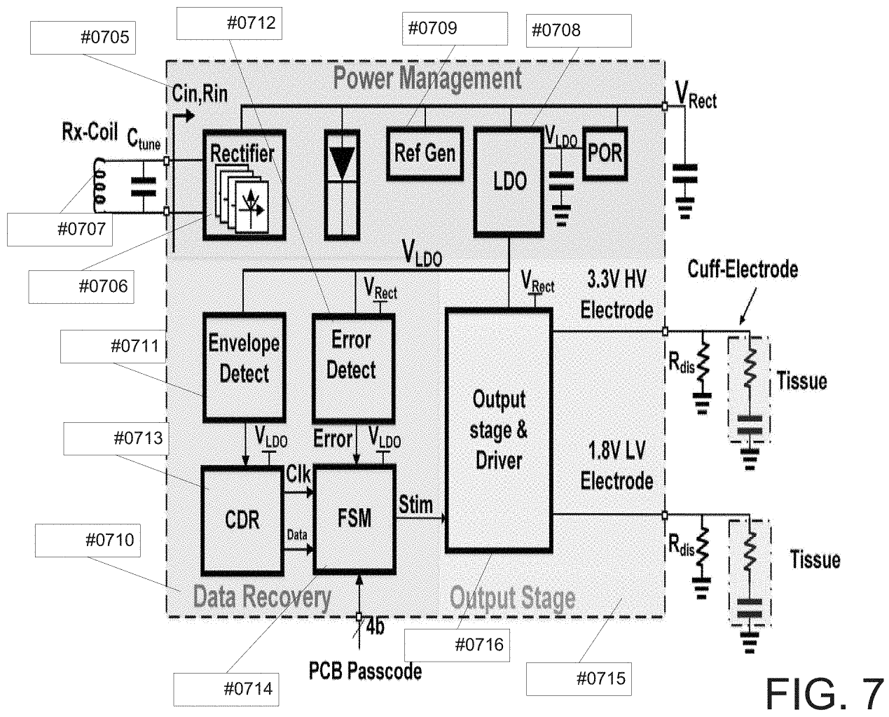

In many embodiments, to ensure batteryless and wireless operation of an implantable device, a power harvesting chip can extract a passcode and control a stimulation. A chip can be interfaced with an Rx coil to harvest the power and extract the clock and data. Tx can send data at a particular rate (e.g., rate of 10 kbps) with PWM-ASK modulation. Based on the pulse width, the clock and data can be extracted. Passcode detection can be realized using a finite state machine (FSM) with received data, and stimuli voltage, amplitude, and duration can be set. An output stage can buffer the control signals and can drive the tissue using standard cuff-electrodes. An on-chip error detection block can ensure that the harvested voltage is not lower than a threshold voltage (e.g., 2.6 V) during stimulations. An architecture of an implantable chip in accordance with an embodiment is illustrated in FIG. 7. As illustrated in FIG. 7, the implantable chip can include a power management unit 705, a data recovery unit 710, and an output stage 715.

In many embodiments, power management unit 705 can be important for an implantable device to operate reliably. A rectifier 706 can harvest power from an Rx coil 707 and can store them on an off-chip capacitor (C_{Str}). An LDO 708 and reference generation 709 can also be important for reliable and efficient operation.

The data recovery block 710 can include an envelope detector 711, error detector 712, CDR 713 and a finite state machine FSM 714, where the FSM can receive a PCB passcode.

The output stage 715 can include output stage and driver circuitry 716 that generate a stimulation on an electrode. Described are further details on different components of implantable stimulator systems in accordance with several embodiments of the invention. Although FIG. 7 illustrates a particular architecture of an implantable stimulation chip, any of a variety of architectures can be utilized as appropriate to the requirements of specific applications in accordance with embodiments of the invention.

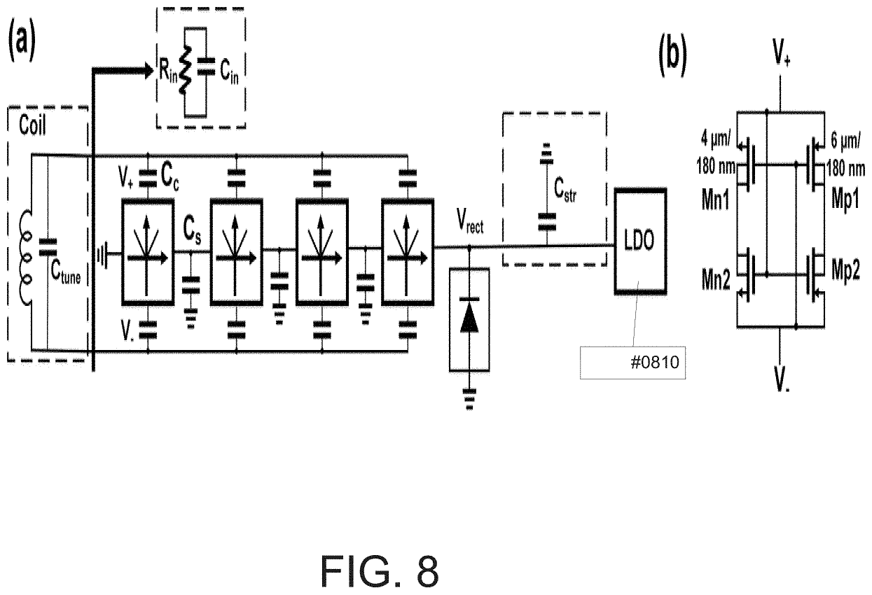

Stimulation systems in accordance with many embodiments can include passive rectifiers that can provide an advantage of efficient cold start-up. Many embodiments of the system can use a cross-coupled (CC) full-wave topology due to its excellent power transfer efficiency and dynamic threshold voltage (Vₜₕ) compensation. A rectifier in accordance with an embodiment of the invention is illustrated in FIG. 8 item (a). A limiter can set a maximum harvested voltage to be a particular voltage (e.g., 3.8 V), and an LDO 810 can provide a stable voltage (e.g., 1.8 V) when the rectifier voltage reaches a level (e.g., 1.8 V). A single stage of a CC rectifier in accordance with an embodiment is illustrated in FIG. 8 item (b). The current in the positive cycle can flow through Mₚ₁ and Mₙ₂, and during the negative cycle, they can flow through Mₚ₂ and Mₙ₁. The nMOS and pMOS can be scaled with a constant ratio according to their mobility. The compensation capacitor can be set to 0.5 pF to compensate for the mismatches between the transistors. The coupling capacitor can enable dynamic gate biasing of the CC rectifier which needs to be large enough (C_{c}=6 pF) so that there is no voltage drop across the capacitor. A challenge of a rectifier design can be choosing the number of stages and sizing of the transistors. The higher number of stages and transistor widths can lead to higher BW while the sensitivity of the rectifier is reduced. As shown in FIG. 8 item (a) in accordance with an embodiment, the rectifier impedance can be presented with parallel resistance (Rᵢₙ) and capacitance (Cᵢₙ). Although FIG. 8 illustrates a particular architecture of a rectifier, any of a variety of architectures can be utilized as appropriate to the requirements of specific applications in accordance with embodiments of the invention.

A quality factor can be expressed based on (2), and for a properly designed Rx coil, the power going to the rectifier can be much larger than the power wasted in the coil, therefore

$${Q}_{R e c t} << {Q}_{R x} = \frac{{L}_{R x} ω}{{R}_{R x}} \qquad ( 6 )$$

$${Q}_{L} \approx {Q}_{R e c t} = \frac{{R}_{i n}}{{L}_{R x} ω} \qquad ( 7 )$$

And therefore, its bandwidth can be calculated from the following equation:

$$B W = \frac{{f}_{0}}{{Q}_{R e c t}} \qquad ( 8 )$$

The inherent efficiency of the rectifier can be another important parameter which is defined as:

$${η}_{R e c t} = \frac{{P}_{D c}}{{P}_{R F}} \qquad ( 9 )$$

The sensitivity of the rectifier can be calculated as Qη_{Rect}.

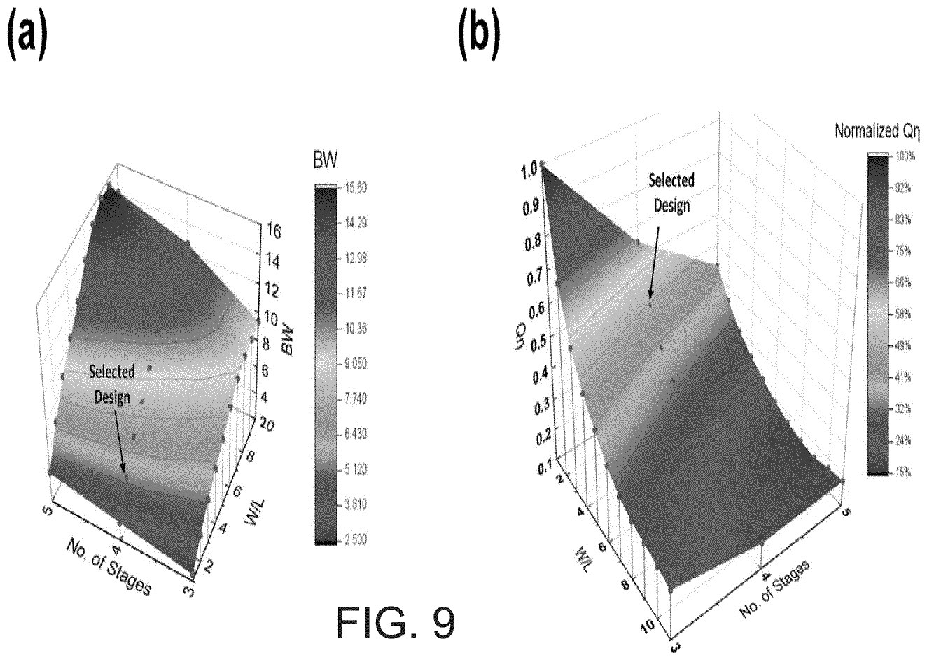

Simulated results of rectifier BW and Qη_{Rect} when the rectifier is loaded by 10 μA of current in accordance with an embodiment are shown in FIG. 9 item (a) and FIG. 9 item (b), respectively. It should be noted that while scaling the width of the transistors, the constant ratio is preserved. In many embodiments, four stages of the CC rectifier with W/L=4 μm/180 nm can be chosen for the nMOS transistors. This choice results in BW of 5.377 MHz and Qη_{Rect} of 0.486.

A resonance frequency of the Rx coil can be calculated using the equation below:

$${f}_{Res} = \frac{1}{2 π \sqrt{( {C}_{i n} + {C}_{t u n e} ) {L}_{R x}}} \qquad ( 10 )$$

In many embodiments, a rectifier can be a non-linear block, and its input impedance varies with power and loading.

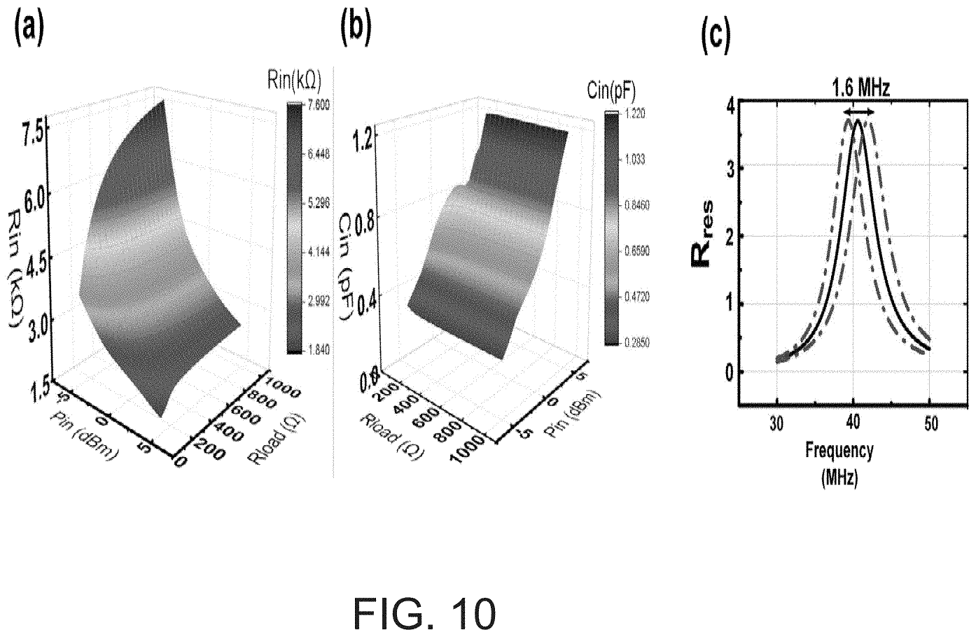

FIG. 10 illustrates variation of a rectifier with respect to input power and loading: (a) Rᵢₙ (b) Cᵢₙ (c) f_{Res} in accordance with an embodiment. As shown in FIG. 10 item (a) and FIG. 10 item (b) in accordance with an embodiment, the input resistance varies with load and input power, and, input capacitance (Cᵢₙ) varies considerably with input power. Variation of input power for a wirelessly powered implant in accordance with many embodiments is inevitable, leading to the variation of the resonance frequency (f_{Res}) based on (10). FIG. 10 item (c) shows the 1.6 MHz shift in f_{Res} due to non-linear input capacitance in accordance with an embodiment. In many embodiments, BW can be chosen wide enough, so resonance resistance does not drop more than a percentage (e.g., 15%) of its nominal value.

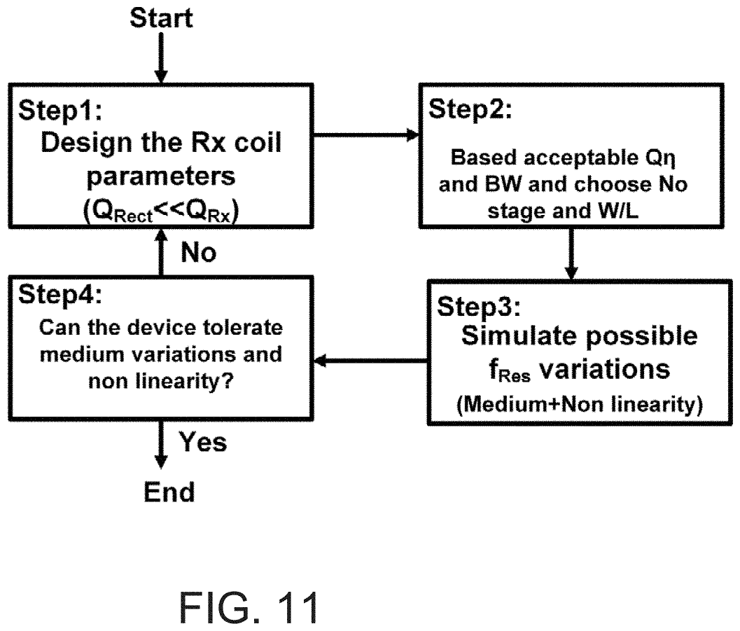

A process of co-designing an Rx coil and rectifier in accordance with an embodiment of the invention is illustrated in FIG. 11. In many embodiments, the Tx frequency is constant, and the goal is to design an Rx coil and rectifier such as it can work at different mediums and power levels. An initial design of an Rx coil in accordance with several embodiments is described herein. It may need to be verified that the coil satisfies (6). In the next step, a reasonable value for the rectifier's efficiency (50%) and BW (3 MHz) can be chosen. In the third step, possible variations, including adding biological tissue and rectifier non-linearity, can be simulated. Lastly, it is verified that a rectifier can tolerate these variations without losing more than a percentage (e.g., 50%) of its performance. An Rx coil may need to be redesigned for a higher BW if the variations cannot be tolerated. The high available BW at the same Q can be an advantage of choosing 40.68 MHz compared to the lower frequency bands (ex: 13.56 MHz). However, it can be more challenging to satisfy SAR regulations due to higher tissue absorption, as shown in FIG. 4 in accordance with an embodiment.

### LDO

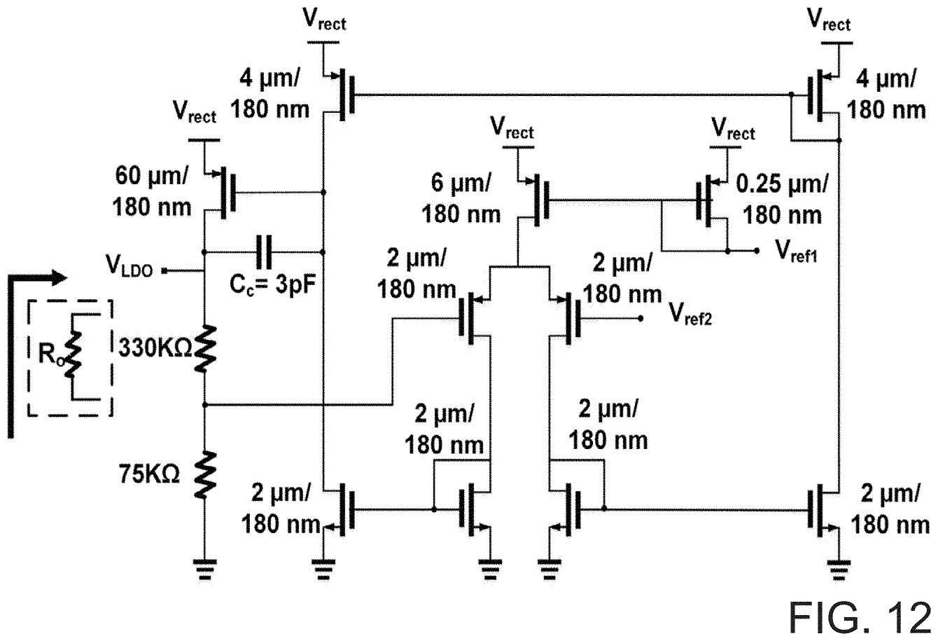

Stimulator systems in accordance with many embodiments can include low drop-out (LDO) regulators that can be important to a chip as they can provide a stable voltage supply (e.g., 1.8 V supply) for other blocks. A schematic of an LDO in accordance with an embodiment of the invention is illustrated in FIG. 12. V_{ref1} and V_{ref2} can be set to 2.64 V and 0.33 V, respectively; an architecture of the reference generator can be based on prior configurations disclosed in the literature. A two-stage LDO can include an error amplifier and a pass transistor as a controlled current source. This structure can benefit from higher loop gain, and a lower voltage drop-out than a source follower pass transistor. A drawback of this structure can be the low phase margin which can cause ringing issues. The trade-off between BW and phase margin can be established depending on the LDO output capacitance. Miller compensation capacitance (C_{c}) of 3 pF can be added to improve the phase margin. In many embodiments, the LDO can have a loop gain of 34.1 dB and a phase margin of 89.94 at 62.3 Hz while loaded with a 1 μF off-chip capacitor. Important features of an LDO in accordance with several embodiments are summarized in table 2. The quiescent power consumption of the LDO (I_{Q}) can be the amount of current the LDO takes at no-load conditions. Line regulation can be an important feature of merit for LDO, defined as:

$$Line Regulation = \frac{Δ {V}_{L D O}}{Δ {V}_{R e c t}} \times 100 \% \qquad ( 11 )$$

Line regulation identifies the ratio between regulated voltage and supply voltage variations. Load Regulation can be defined as the amount of the LDO voltage drop concerning a change in the loading current. The output resistance of the LDO can be tightly related to the load regulation but in a small signal domain:

$${R}_{o n} = \frac{δ {V}_{L D O}}{δ {I}_{l o a d}} \qquad ( 12 )$$

Although FIG. 12 illustrates a particular architecture of an LDO, any of a variety of architectures can be utilized as appropriate to the requirements of specific applications in accordance with embodiments of the invention.

TABLE II
LDO SPECIFICATIONS

| | LDO |
|---|---|
| I_{Q} | 7.6 μA |
| Rₒₙ | 30 Ω |
| Line Reg | 1.8% |
| Load Reg | 60 mV @ 1 mA |

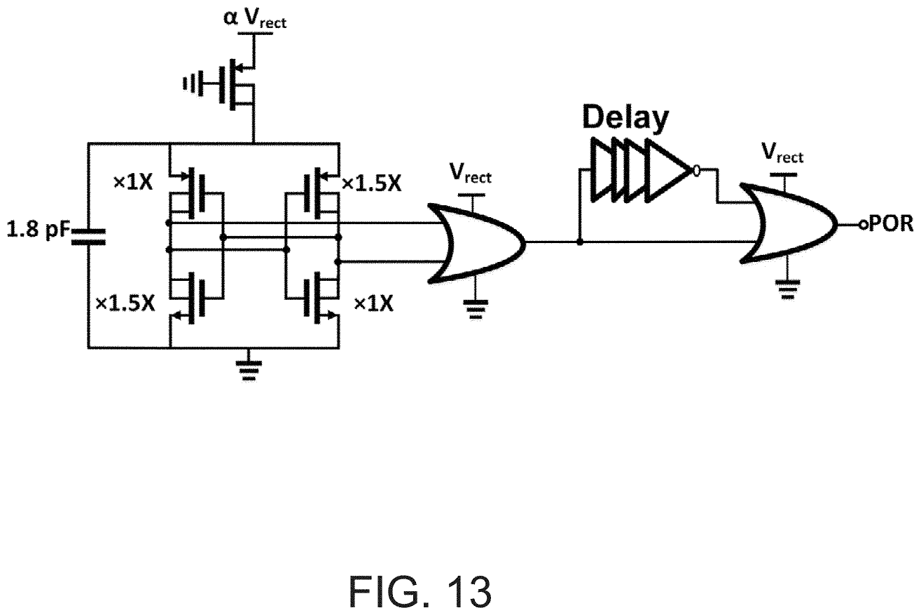

It can be important for a wirelessly powered device to reset the digital blocks every time the chip is powered up. A cross-coupled inverter chain can be used for POR. A fraction of rectifier voltage (α=0.75) can be used for a weak current source to charge the capacitor in cross-coupled inverters. The inverters can be sized for opposite pull-up and pull-down to prevent metastability. As shown in FIG. 13, the generated pulse can be further extended by capacitively loaded inverter chain to 50 ns. The digital blocks can be reset every time the harvested voltage reaches 0.9 V. Although FIG. 13 illustrates a particular architecture of a cross-coupled inverter chain, any of a variety of architectures can be utilized as appropriate to the requirements of specific applications in accordance with embodiments of the invention.

### Clock and Data Recovery

In many embodiments, clock and data recovery can be used to determine a stimulation's passcode, duration, and/or voltage level. Unlike power-hungry phase detectors, the difference between the pulse width of bit ‘1’ and ‘0’ can be utilized to recover the clock and data. In many embodiments, bits ‘1’ and ‘0’ can have pulse widths of 75% and 25% respectively, and the duration of each bit can be set to be 100 μs. The signal's envelope can be used as the clock for the system.

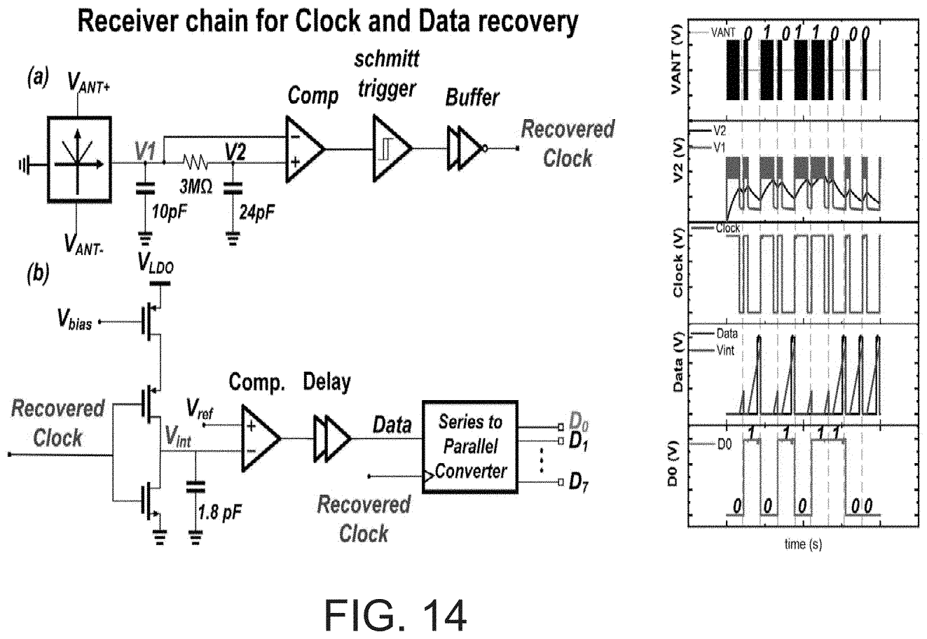

FIG. 14 illustrates a circuit architecture for clock and data recovery, in particular FIG. 14 item (a) illustrates clock recovery chain and FIG. 14 item (b) illustrates data recovery chain in accordance with an embodiment of the invention. FIG. 14 illustrates that one stage of the cross-coupled rectifier can operate as a self-mixer. The signal at the antenna side (V_{ANT}) is passed through a self-mixer (V₁) and low pass filtered (V₂) with a corner frequency of 2.1 kHz. The 10 pF capacitor at the output of the mixer is to filter out the 2f_{RF} frequency. The comparator can be used in the next stage to remove the DC components. Self-mixing action can be expressed using the equations below:

$${V}_{A N T} = A [ 1 - m x ( t ) ] cos ( 2 π {f}_{R F} t ) \qquad ( 13 )$$

$${V}_{1} \approx A [ 1 - m x ( t ) ] cos ( 2 π {f}_{R F} t ) \times \frac{2}{π} cos ( 2 π {f}_{R F} t ) \qquad ( 14 )$$

$${V}_{1 , LPF} = \frac{A}{π} [ 1 - m x ( t ) ] \qquad ( 15 )$$

In FIG. 14, the simulation for OOK (m=100%) is illustrated. However, to save power and maintain a good PTE during data transfer, m can reduce to 5%. To increase stability and noise robustness, a Schmitt trigger can be added. Although FIG. 14 illustrates a particular circuit architecture for clock and data recovery, any of a variety of architectures can be utilized as appropriate to the requirements of specific applications in accordance with embodiments of the invention.

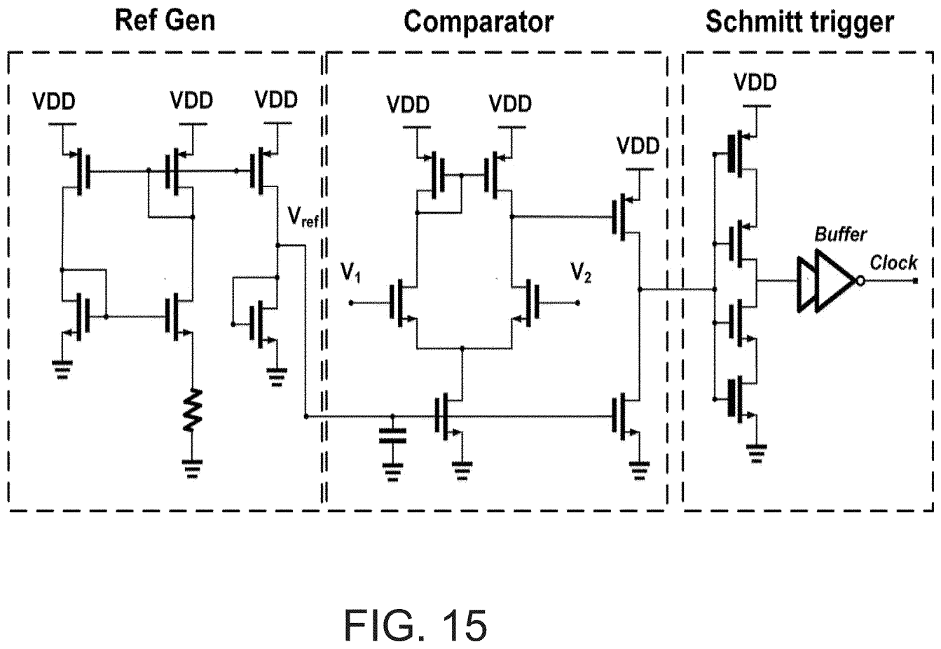

A circuit schematic of a reference generator, comparator and Schmitt trigger in accordance with an embodiment of the invention is illustrated in FIG. 15. The comparator can have a gain of 51 dB with 4 MHz bandwidth, and the Schmitt trigger can have a hysteresis window of 0.35 V to 0.95 V. A reference generator can be used due to fast start-up and temperature stability. Although FIG. 15 illustrates a particular circuit architecture of reference generator, comparator, and Schmitt trigger, any of a variety of architectures can be utilized as appropriate to the requirements of specific applications in accordance with embodiments of the invention.

In many embodiments, the clock can be integrated by a 40 nA current source on a 1.8 pF capacitor. Bit ‘1’ and ‘0’ reach values of 1.65 V and 0.375 V after integration (Vᵢₙₜ). After comparing with the reference voltage of 1.6 V, a pulse of 18 μs is generated if the bit is ‘1’. In order to avoid glitches, a delay of 6 μs can be applied for the detected pulses, then they are passed through a shift register to generate the data (D₀) for passcode detection and stimulation. This scheme can enable clock and data recovery without phase locked loops.

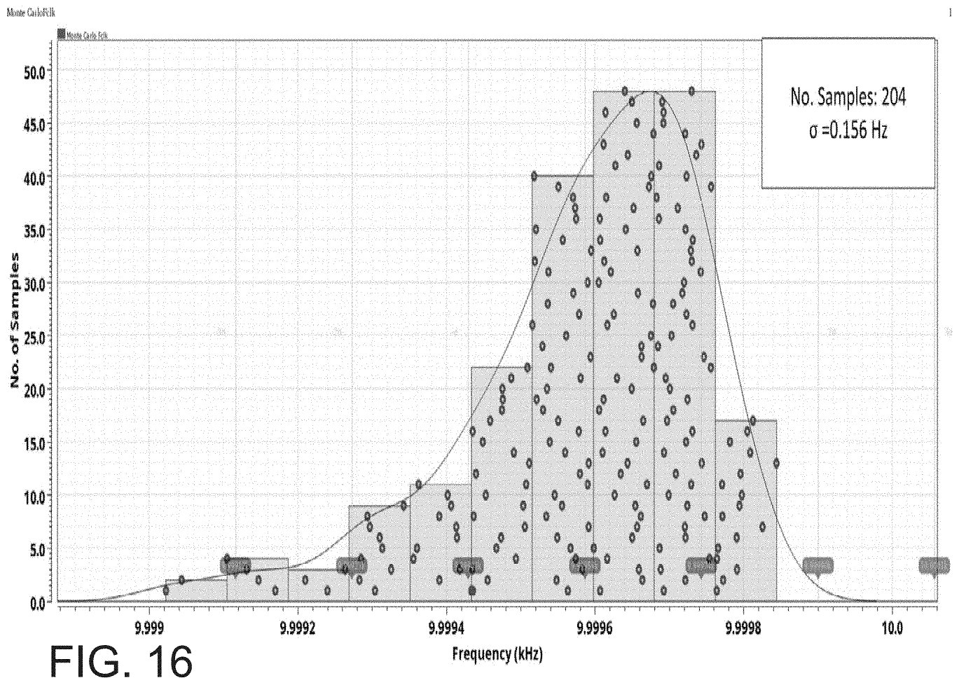

Monte Carlo over 204 post-layout simulations can be performed. FIG. 16 illustrates Monte Carlo simulation of a recovered clock frequency in accordance with an embodiment of the invention. As shown in FIG. 16, the standard deviation (a) of the recovered signal frequency is 0.154 Hz which shows accurate clock recovery with process variations.

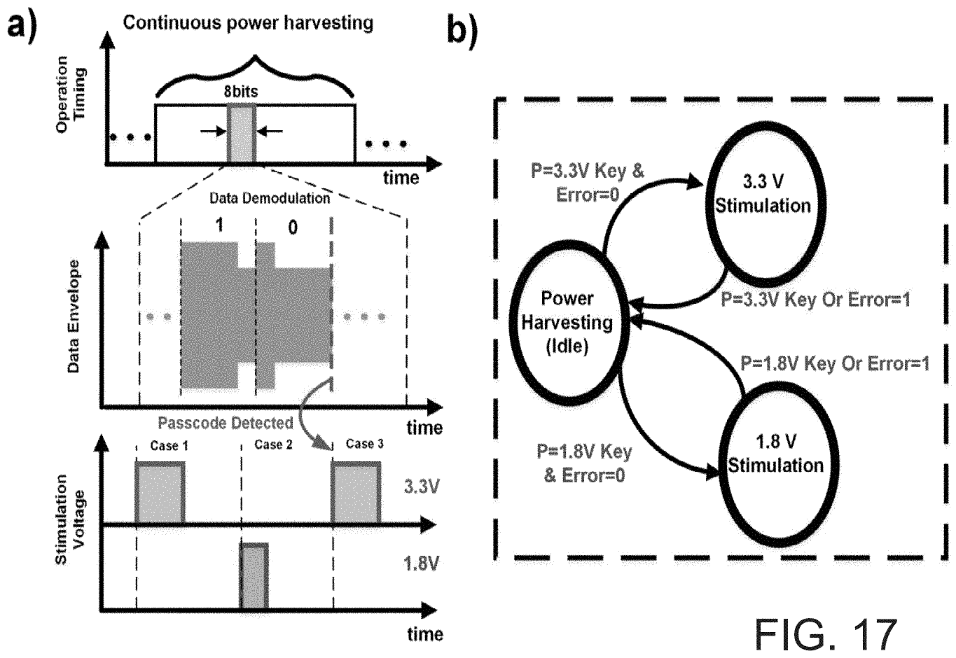

In many embodiments, each chip can have a specific n-bit (e.g., 4-bit) passcode based on the PCB connections. After sending n bits (e.g., 8 bits), including flag and voltage level, if the passcode and flag are matched, the stimulation activates. FIG. 17 item (a) illustrates a timing diagram of a chip and how independent channels stimulate in accordance with an embodiment of the invention. FIG. 17 item (b) illustrates the FSM for stimulation in accordance with an embodiment of the invention. The error signal generated for the output stage verifies if enough voltage is harvested on the storage capacitor and stimulation happens only when the error signal is zero. Although FIG. 17 illustrates a particular design of an FSM, any of a variety of FSM designs for stimulation can be utilized as appropriate to the requirements of specific applications in accordance with embodiments of the invention.

### Output Stage

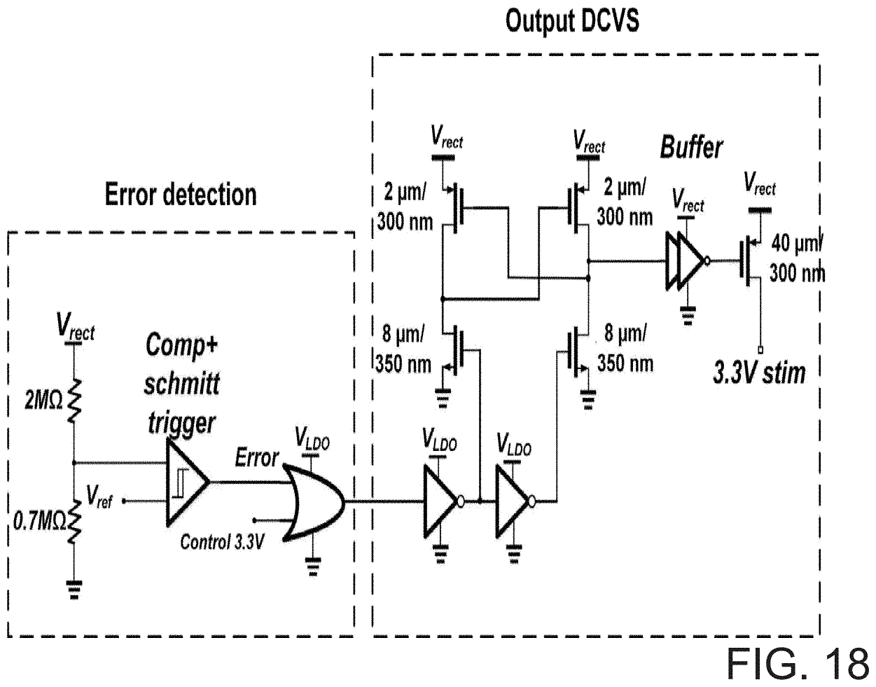

An output stage of a stimulator in accordance with an embodiment of the invention is illustrated in FIG. 18. An error detection block can stop the stimulation if the harvested voltage drops below a threshold (e.g., 2.6 V). In many embodiments, a conventional differential cascode voltage switch (DCVS) can be used due to its low static power consumption and fast switching. DCVS can shift a control signal from different voltage (e.g., 1.8 V to 3.3 V) domain. NMOS transistors can be scaled up to ensure a strong pull-down network. The signal after buffering can have a 1 ns delay. The static and dynamic power (f=10 KHz) consumption of the output stage with buffers is 30 pW and 155 nW, respectively.

The minimum tolerable load resistance for the storage capacitor can be defined as:

$${R}_{load , min} = \frac{{V}_{S tim , Avg} \times {t}_{S t i m}}{{C}_{s t r} \times Δ {V}_{s t i m}} \qquad ( 16 )$$

Where V_{Stim,Avg} is the average stimulation voltage, t_{Stim} is the stimulation pulse time, Cₛₜᵣ is the storage capacitance, and ΔVₛₜᵢₘ is the maximum allowable voltage change during stimulation. For a conventional stimulation of t_{Stim}=1 ms, V_{Stim,Avg}=3.3 V and ΔVₛₜᵢₘ=0.3 V, the R_{load,min} is 271Ω. The maximum load for stable stimulation may need to be verified to be within operating range.

Although FIG. 18 illustrates a particular circuit architecture of an output stage of an implantable stimulator, any of a variety of circuit architectures can be utilized as appropriate to the requirements of specific applications in accordance with embodiments of the invention.

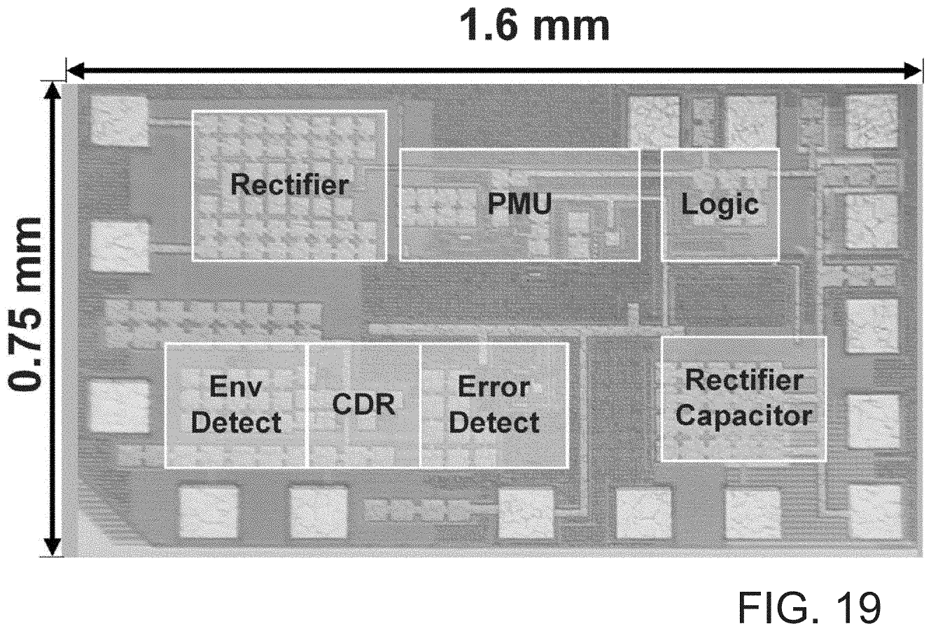

In many embodiments, a SoC can be fabricated in TSMC 180-nm technology. A chip can have an area of 1.2 mm2, as shown in FIG. 19 in accordance with an embodiment of the invention.

Although specific implementations for code-controlled multi-side wirelessly powered battery-less implantable stimulators are discussed above with respect to FIGS. 1-19, any of a variety of implementations utilizing the above discussed techniques can be utilized code-controlled multi-site wirelessly powered battery-less implantable stimulators in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A wirelessly powered and controlled implantable stimulator system, comprising:
a transmitter (TX) coil wirelessly powering and controlling a plurality of implantable stimulators though electromagnetic waves, wherein the electromagnetic waves comprise modulated waveforms that include n-bit passcodes to individually control stimulation of each of the plurality of implantable stimulators;
wherein an implantable stimulator of the plurality of implantable stimulators comprises:
a receiver (RX) for receiving a modulated waveform from the TX coil, wherein the implantable stimulator is controlled based on the modulated waveform;
a coil that harvests AC power;
a voltage regulator;
a rectifier that rectifies the harvested power and passes the rectified voltage (Vrect) to the voltage regulator to provide a constant voltage;
data recovery and control circuitry coupled to the voltage regulator and configured to output a stimulation control signal based on passcodes detected from the modulated waveform;
an output stage and driver circuitry coupled to receive the constant voltage from the voltage regulator and the stimulation control signal from the data recovery and control circuitry, and configured to output a stimulation signal; and
one or more electrodes coupled to receive the stimulation signal from the output stage and driver circuitry.

2. The implantable stimulator system of claim 1, wherein an n-bit passcode is hard-wired and set on a printed circuit board (PCB) for each of the plurality of implantable stimulators, wherein the modulated waveform comprises an n-bit passcode to communicate with a particular implantable stimulator of the plurality of implantable stimulators, wherein the modulated waveform comprises a plurality of stimulation control settings including a start/stop signal and a voltage level of a stimulation pulse to be delivered by an implantable stimulator.

3. The implantable stimulator system of claim 1, wherein the modulated waveform comprises a plurality of bits, wherein a first set of bits of the plurality of bits address a particular implantable stimulator from the plurality of stimulators, at least one bit of the plurality of bits determines a voltage level of a stimulation, and at least one bit of the plurality of bits determines a start/stop signal of a stimulation.

4. The implantable stimulator system of claim 1, wherein the TX coil wirelessly powers and controls the plurality of implantable stimulators through a near field resonant inductive link.

5. The implantable stimulator of claim 1, wherein the modulated waveform is a pulse-width modulated amplitude-shift keying (PWM-ASK) waveform.

6. The implantable stimulator system of claim 1, wherein the Vrect drops below a threshold voltage and a control block stops stimulation.

7. The implantable stimulator system of claim 1, wherein at least one implantable stimulator in the plurality implantable stimulators is battery-less such that the implantable stimulator is wirelessly powered and memory-less such that control of a stimulation of the at least one implantable stimulator is controlled based on the modulated waveform received from the TX coil.

8. The implantable stimulator system of claim 1, wherein each implantable stimulator in the plurality of implantable stimulators is individually addressable using an n-bit passcode.

9. The implantable stimulator system of claim 1, wherein the implantable stimulator further comprises a system on chip (SOC), wherein the SOC comprises:
a power management unit (PMU);
a clock and data recovery (CDR) unit that recovers clock and data from the modulated waveform;
an envelope detection unit;
an error detection unit; and
an output driving stage.

10. The implantable stimulator system of claim 1, wherein the voltage regulator is a low dropout voltage regulator (LDO) comprising an amplifier in a negative feedback loop.

11. The implantable stimulator system of claim 1, wherein the Vrect exceeds a threshold voltage and a limiter is activated and stops the Vrect from accumulating further.

12. The implantable stimulator system of claim 1, wherein the rectifier is a fully differential cross-coupled 4-stage rectifier.

13. The implantable stimulator system of claim 1, wherein the RX is used to extract both clock and data, wherein an incoming signal is self-mixed using the rectifier and low-pass filtered and passed through a Schmitt trigger to extract its envelope that includes baseband data.

14. The implantable stimulator system of claim 9, wherein clock and data are recovered by passing the envelope though an integrator and a comparator.

15. The implantable stimulator system of claim 1, wherein the implantable stimulator further comprising a finite state machine (FSM) that controls a stimulation pulse width and amplitude of stimulation.

16. The implantable stimulator system of claim 1, wherein the implantable stimulator is controlled by a 4-bit passcode.

17. A medical system comprising:
a transmitter (TX) coil configured to output transmitter waves having a pulse-width and an amplitude; and
a plurality of implantable stimulators, each implantable stimulator comprising:
a coil configured to establish a near-field resonant inductive link with the transmitter coil, and to harvest power from the transmitter waves;
a voltage regulator that outputs a constant voltage;
data recovery and control circuitry coupled to the voltage regulator and configured to output a stimulation control signal based on passcodes detected from the transmitter waves;
an output stage and driver circuitry coupled to receive the constant voltage from the voltage regulator and the stimulation control signal from the data recovery and control circuitry, and configured to output a stimulation signal; and
one or more electrodes coupled to receive the stimulation signal from the output stage and driver circuitry.

18. The medical system of claim 17, wherein each implantable stimulator further comprises:
a rectifier that rectifies the harvested power and passes the rectified voltage (Vrect) to the voltage regulator to provide the constant voltage;
wherein output stage and driver circuitry is configured to stop stimulation in response to the Vrect dropping below a threshold voltage.

19. The medical system of claim 17, wherein at least one of the plurality of implantable stimulators is configured for implant in or on a heart, and the stimulation signal is configured to evoke a depolarization of the heart.

20. The medical system of claim 17, wherein a first of the plurality of implantable stimulators is configured for implant in or on a left ventricle of a heart, a second of the plurality of implantable stimulators is configured for implant in or on a right ventricle of a heart, and the output stage and driver circuitry is configured to output respective stimulation signals to the first implantable stimulator and the second implantable stimulator to deliver biventricular pacing.

21. The medical system of claim 17, wherein at least one of the plurality of implantable stimulators is configured for implant in or on a brain, and the stimulation signal is configured to elicit a neural effect.

22. The medical system of claim 17, wherein at least one of the plurality of implantable stimulators is configured for implant in or on a spine, and the stimulation signal is configured to elicit a neural effect.

23. The medical system of claim 17, wherein a first of the plurality of implantable stimulators is configured for implant on a first side of a neck to stimulate a right vagus nerve, a second of the plurality of implantable stimulators is configured for implant on a second side of the neck to stimulate a left vagus nerve, and the output stage and driver circuitry is configured to output respective stimulation signals to the first implantable stimulator and the second implantable stimulator.

24. The medical system of claim 23, wherein the nerve is a vagal nerve.

25. The medical system of claim 17, wherein the transmitter (TX) coil is included in a component configured to be implanted in operation vicinity (within about 80 mm) of each of the plurality of implantable stimulators.

26. The medical system of claim 17, wherein the transmitter (TX) coil is included in an external component configured to be placed in operation vicinity (within about 80 mm) of each of the plurality of implantable stimulators.

27. The medical system of claim 17, wherein at least one of the plurality of implantable stimulators is configured for implant in, on, or adjacent to a peripheral nerve.
